(19) United States
(12) Patent Application Publication
(10) Pub. No.: US 2016/0015852 A1
(43) Pub. Date: Jan. 21, 2016

(54) COMPOSITE MATERIAL

(71) Applicant: Industrial Techology Research Institute, Hsinchu (TW)

(72) Inventors: Yu-Bing LIOU, Hsinchu City (TW); Hsiu-Ying WANG, New Taipei City (TW); Hsin-Hsin SHEN, Zhudong Township (TW); Ying-Wen SHEN, Zhunan Township (TW); Chin-Tsung HUANG, Hsinchu City (TW); Hsin-Yi HSU, Taoyuan City (TW)

(73) Assignee: INDUSTRIAL TECHNOLOGY RESEARCH INSTITUTE, Hsinchu (TW)

(21) Appl. No.: 14/799,071

(22) Filed: Jul. 14, 2015

Related U.S. Application Data
(60) Provisional application No. 62/024,626, filed on Jul. 15, 2014.

(30) Foreign Application Priority Data
Dec. 12, 2014 (TW) 103143432

(51) Int. Cl. A61L 15/64; A61L 15/22; A61L 15/24; A61L 15/32; A61L 15/26

(52) U.S. Cl. CPC A61L 15/64; A61L 15/325; A61L 15/26; A61L 15/24; A61L 15/225

(57) ABSTRACT

According to embodiments, a composite material is disclosed. The composite material has a multi-layered structure, wherein the multi-layered structure is constituted by a hydrophilic biodegradable polymer and a collagen. In particular, the collagen is strip-shaped and has a fiber length from 1.5 mm to 50 mm. There are at least ten stacked layers per 5 μm of thickness in the multi-layered structure.

### CROSS REFERENCE TO RELATED APPLICATIONS

This application claims the benefit of U.S. Provisional Application No. 62/024,626, filed on Jul. 15, 2014, which is incorporated herein by reference.

The application is based on, and claims priority from, Taiwan Application Serial Number 103143432, filed on Dec. 12, 2014, the disclosure of which is hereby incorporated by reference herein in its entirety.

### TECHNICAL FIELD

The disclosure relates to a composite material.

### BACKGROUND

Generally, skin wounds should be kept relatively dry so as to facilitate the healing process. Hence, gauze is used conventionally to keep wounds sterile and dry. However, gauze may sometimes adhere to the tissues or exudates of the wound. Such adhesion may result in secondary damage to the tissues surrounding the wound when the gauze is removed.

Recently, it has been established that a moistening environment may facilitate the healing of wounds. The fluids secreted by the wound may contain various growth factors that are advantageous to healing. The conventional water-absorption materials, however, exhibit poor tensile strength, and are not suitable for use with surgical sutures. In addition, due to their poor light transparency, conventional degradable biomaterials interfere with the observation of the wound.

Therefore, a novel degradable material for use in the biomedical field is desired solving the aforementioned problems.

### SUMMARY

According to an embodiment of the disclosure, the disclosure provides a composite material which has a multi-layered structure constituted by a hydrophilic biodegradable polymer and a collagen. In particular, in the multi-layered structure, there are at least ten stacked layers per 5 μm of thickness. Each stacked layer has a thickness between 0.1 to 1 μm. Furthermore, the collagen may be strip-shaped and have a fiber length between about 1.5 mm and 50 mm.

According to another embodiment of the disclosure, the composite material of the disclosure can be a product fabricated by the following steps. A hydrophilic biodegradable polymer is dissolved into a solvent, obtaining a first solution. The pH value of the first solution is adjusted to be lower than or equal to 5. A collagen is added into the first solution, obtaining a second solution, wherein the collagen is strip-shaped and has a fiber length between 1.5 mm and 50 mm. The second solution is subjected to a drying process, obtaining a film.

A detailed description is given in the following embodiments with reference to the accompanying drawings.

### BRIEF DESCRIPTION OF THE DRAWINGS

The disclosure may be more fully understood by reading the subsequent detailed description and examples with references made to the accompanying drawings, wherein:

FIG. 1 shows a scanning electron microscope (SEM) image of the film (V) of Example 5.

FIG. 2 shows a scanning electron microscope (SEM) image of the film (VII) of Example 7.

### DETAILED DESCRIPTION

The following description is of the best-contemplated mode of carrying out the disclosure. This description is made for the purpose of illustrating the general principles of the disclosure and should not be taken in a limiting sense. The scope of the disclosure is best determined by reference to the appended claims.

The disclosure provides a composite material prepared from subjecting a hydrophilic biodegradable polymer and a collagen to a drying process. Since the collagen has a longer fiber length, the composite material can have a multi-layered structure, resulting in the composite material exhibiting a high water-absorption ability. In particular, in the multi-layered structure, there are at least ten stacked layers per 5 μm of thickness. Each stacked layer has a thickness between 0.1 to 1 μm. In addition, the composite material of the disclosure can exhibit a high suture pull-out strength and high light transmittance after swelling with water (i.e. wet film). Furthermore, the composite material of the disclosure can be applied to wound dressing, ophthalmology, orthopedic implants, surgical use, drug delivery, or tissue engineering.

The composite material of the disclosure can have a multi-layered structure constituted by a hydrophilic biodegradable polymer and a collagen, wherein the collagen is strip-shaped and has a fiber length between about 1.5 mm and 50 mm, such as between about 15 mm and 30 mm.

The collagen fibers can be in a fully extended state due to the intramolecular charge repulsion and the hydrogen bond interaction between the collagen and water, when placed in an acid solution. Meanwhile, due to the fiber length being longer than 1.5 mm (i.e. strip-shaped fiber, rather than flocculent fiber), the collagen fiber can be stacked regularly during drying, resulting in a composite material having a multi-layered structure. In particular, in the multi-layered structure, there are at least ten stacked layers per 5 μm of thickness. Each stacked layer has a thickness between 0.1 to 1 μm.

According to embodiments of the disclosure, the hydrophilic biodegradable polymer includes polyvinyl alcohol (PVA), polyethylene glycol/polyethylene oxide (PEG/PEO), polyvinylpyrrolidone (PVP), or a combination thereof. The hydrophilic biodegradable polymer can have a molecular weight between about 300 and 1,500,000. The degradation rate of the composite material can be controlled by modifying the molecular weight of the hydrophilic biodegradable polymer. For example, the hydrophilic biodegradable polymers with a relatively low molecular weight (such as between 300 and 60,000) results in a high degradation rate of the composite material. On the other hand, the hydrophilic biodegradable polymers with a relatively high molecular weight (such as between 100,000 and 1,500,000) results in a slow degradation rate of the composite material. For example, when the hydrophilic biodegradable polymer is polyvinyl alcohol (PVA), the hydrophilic biodegradable polymer can have a molecular weight between about 10,000 and 130,000; when the hydrophilic biodegradable polymer is polyethylene glycol/polyethylene oxide (PEG/PEO), the hydrophilic biodegradable polymer can have a molecular weight between about 300 and 150,000; and, when the hydrophilic biodegradable polymer is polyvinylpyrrolidone (PVP), the hydrophilic biodegradable polymer can have a molecular weight between about 10,000 and 1,500,000.

According to embodiments of the disclosure, the weight ratio between the collagen and the hydrophilic biodegradable polymer can be from 1:3 to 9:1, such as from 1:3 to 3:1, or from 1:1 to 4:1. When the weight ratio of the collagen and hydrophilic biodegradable polymer is too low, the composite material is relatively brittle and apt to dissolve in water (rather than forming a film) due to the absence of collagen fibers. On the other hand, when the weight ratio of the collagen and hydrophilic biodegradable polymer is too high, the light transmittance and the swelling ratio of the composite material are reduced.

According to some embodiments of the disclosure, the method for fabricating the composite material of the disclosure can include following steps. First, a hydrophilic biodegradable polymer is dissolved into a solvent, obtaining a first solution. Next, the pH value of the first solution is adjusted to be lower than or equal to 5, such as lower than or equal to 3. The subsequently added collagen can be completely dissolved in the solvent when the first solution has a pH value lower than or equal to 5. When the pH value of the first solution is larger than 5, the collagen would be separated out rather than dissolving in the solvent. Next, a collagen is added into the first solution, obtaining a second solution, wherein the collagen is strip-shaped and has a fiber length between 1.5 mm and 50 mm. Next, the second solution is subjected to a drying process, obtaining a film. Since there is a high miscibility between the collagen and the hydrophilic biodegradable polymer, the drying process can be a biaxial stretching process or solvent casting.

According to embodiments of the disclosure, after the drying process, the film can be subject to a treatment so that the hydrophilic biodegradable polymer and/or the collagen undergoes a cross-linking reaction. The cross-linking reaction can effectively increase the degradation period of the composite material. The treatment can be a chemical cross-linking process with a cross-linking agent. The cross-linking agent can include formaldehyde, glutaraldehyde, glyoxal, malondialdehyde, succinyl dialdehyde, phthalaldehyde, dialdehyde starch, polyacrolein, polymethacrolein, or a combination thereof. Due to the use of aldehyde as a cross-linking agent, the collagen of the composite material can be further cross-linked via the chemical cross-linking process.

According to another embodiment of the disclosure, the treatment can be a physical cross-linking process. In the physical cross-linking process, the film is irradiated by radiation, wherein the radiation can be an ultraviolet light, or a Gamma ray. When the composite material is irradiated with ultraviolet light, the collagen and hydrophilic biodegradable polymer of the composite material can be cross-linked further. The cross-linking reaction can have a reaction time from 10 minutes to several hours. The cross-linking degree of the composite material is proportional to the reaction time of the cross-linking reaction, and the degradation rate of the composite material is inversely proportional to the reaction time of the cross-linking reaction.

According to embodiments of the disclosure, the composite material of the disclosure can have a swelling ratio between about 1 and 15 (such as between about 2 and 15), a light transmittance greater than or equal to 90%, and a suture pull-out strength between about 3 Mpa and 50 Mpa.

Below, exemplary embodiments will be described in detail with reference to the accompanying drawings so as to be easily realized by a person having ordinary knowledge in the art. The concept of the disclosure may be embodied in various forms without being limited to the exemplary embodiments set forth herein. Descriptions of well-known parts are omitted for clarity, and like reference numerals refer to like elements throughout.

### Fabrication of the Film

### Example 1

First, 0.5 g of collagen (strip-shaped fiber with a fiber length about 15 mm), and 100 mL of an aqueous solution (pH<5) were added into a reaction bottle. Next, after the collagen was dissolved into the water, the solution was injected into a two-dimensional mold and dried at room temperature. Next, the result was disposed in a chamber under the saturated vapor pressure of formaldehyde for 1 hour to undergo a cross-linking reaction, obtaining a film (I).

### Example 2

First, 0.5 g of collagen (strip-shaped fiber with a fiber length about 15 mm), and 100 mL of an aqueous solution (pH<5) were added into a reaction bottle. Next, after the collagen was dissolved into the water, the solution was injected into a two-dimensional mold and dried at room temperature. Next, the result was disposed in a chamber and irradiated by an ultraviolet light with a wavelength of 254 nm and an intensity of 3 mW/cm² for 1 hour to undergo a cross-linking reaction, obtaining a film (II).

### Example 3

First, 0.5 g of polyvinyl alcohol (PVA, with a molecular weight about 30,000-50,000), and 100 mL of water were added into a reaction bottle. Next, after the collagen was dissolved into the water, HCl aqueous solution (6N) was added into the reaction bottle, obtaining a solution with a pH lower than 3. Next, 0.5 g of collagen (strip-shaped fiber with a fiber length about 15 mm) was added into the reaction bottle. The weight ratio of the collagen and hydrophilic biodegradable polymer was 1:1. Next, after the collagen was dissolved into the water, the solution was injected into a two-dimensional mold and dried at room temperature. Next, the result was disposed in a chamber under the saturated vapor pressure of formaldehyde for 1 hour to undergo a cross-linking reaction, obtaining a film (III).

### Example 4

First, 0.5 g of polyethylene glycol/polyethylene glycol (PEG/PEO, with a molecular weight about 30,000-70,000), and 100 mL of water were added into a reaction bottle. Next, after the collagen was dissolved into the water, HCl aqueous solution (6N) was added into the reaction bottle, obtaining a solution with a pH lower than 3. Next, 0.5 g of collagen (strip-shaped fiber with a fiber length about 15 mm) was added into the reaction bottle. The weight ratio of the collagen and hydrophilic biodegradable polymer was 1:1. Next, after the collagen was dissolved into the water, the solution was injected into a two-dimensional mold and dried at room temperature. Next, the result was disposed in a chamber under the saturated vapor pressure of formaldehyde for 1 hour to undergo a cross-linking reaction, obtaining a film (IV).

### Example 4-1

First, 0.17 g of polyethylene glycol/polyethylene glycol (PEG/PEO, with a molecular weight about 300-1000), and 100 mL of water were added into a reaction bottle. Next, after the collagen was dissolved into the water, HCl aqueous solution (6N) was added into the reaction bottle, obtaining a solution with a pH lower than 3. Next, 0.5 g of collagen (strip-shaped fiber with a fiber length about 15 mm) was added into the reaction bottle. The weight ratio of the collagen and hydrophilic biodegradable polymer was 3:1. Next, after the collagen was dissolved into the water, the solution was injected into a two-dimensional mold and dried at room temperature. Next, the result was disposed in a chamber under the saturated vapor pressure of formaldehyde for 1 hour to undergo a cross-linking reaction, obtaining a film.

### Example 5

First, 0.5 g of polyvinylpyrrolidone (PVP, with a molecular weight about 50,000-60,000), and 100 mL of water were added into a reaction bottle. Next, after the collagen was dissolved into the water, HCl aqueous solution (6N) was added into the reaction bottle, obtaining a solution with a pH lower than 3. Next, 0.5 g of collagen (strip-shaped fiber with a fiber length about 15 mm) was added into the reaction bottle. The weight ratio of the collagen and hydrophilic biodegradable polymer was 1:1. Next, after the collagen was dissolved into the water, the solution was injected into a two-dimensional mold and dried at room temperature. Next, the result was disposed in a chamber under the saturated vapor pressure of formaldehyde for 1 hour to undergo a cross-linking reaction, obtaining a film (V).

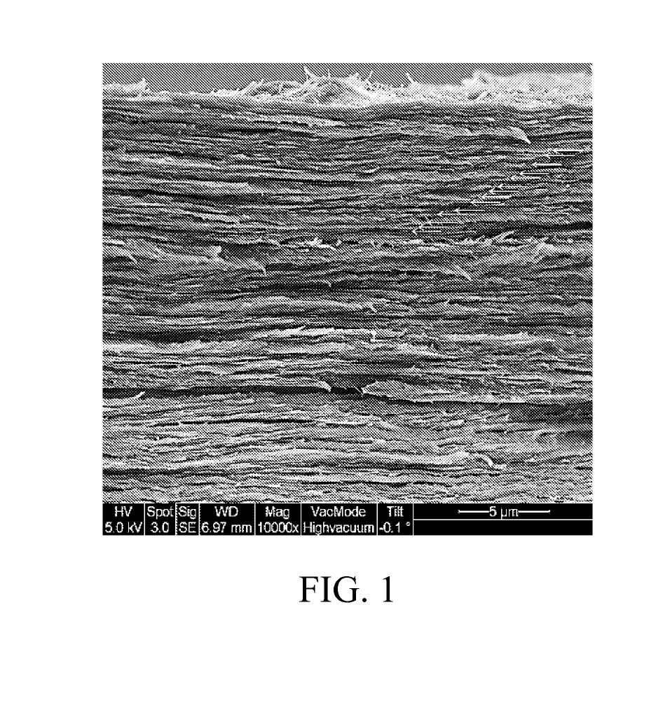

The film (V) was observed by a scanning electron microscope, and the result is shown in FIG. 1, wherein arrows in FIG. 1 point out the stacked layers. As shown in FIG. 1, the film (V) has a multi-layered structure, and there are at least ten stacked layers per 5 μm of thickness. Furthermore, each stacked layer has a thickness between 0.1 to 1 μm.

### Example 5-1

First, 0.5 g of polyvinylpyrrolidone (PVP, with a molecular weight about 300,000-400,000), and 100 mL of water were added into a reaction bottle. Next, after the collagen was dissolved into the water, HCl aqueous solution (6N) was added into the reaction bottle, obtaining a solution with a pH lower than 3. Next, 0.5 g of collagen (strip-shaped fiber with a fiber length about 15 mm) was added into the reaction bottle. The weight ratio of the collagen and hydrophilic biodegradable polymer was 1:1. Next, after the collagen was dissolved into the water, the solution was injected into a two-dimensional mold and dried at room temperature. Next, the result was disposed in a chamber under the saturated vapor pressure of formaldehyde for 1 hour to undergo a cross-linking reaction, obtaining a film

### Example 5-2

First, 1.5 g of polyvinylpyrrolidone (PVP, with a molecular weight about 50,000-60,000), and 100 mL of water were added into a reaction bottle. Next, after the collagen was dissolved into the water, HCl aqueous solution (6N) was added into the reaction bottle, obtaining a solution with a pH lower than 3. Next, 0.5 g of collagen (strip-shaped fiber with a fiber length about 15 mm) was added into the reaction bottle. The weight ratio of the collagen and hydrophilic biodegradable polymer was 1:3. Next, after the collagen was dissolved into the water, the solution was injected into a two-dimensional mold and dried at room temperature. Next, the result was disposed in a chamber under the saturated vapor pressure of formaldehyde for 1 hour to undergo a cross-linking reaction, obtaining a film.

### Example 5-3

First, 0.125 g of polyvinylpyrrolidone (PVP, with a molecular weight about 50,000-60,000), and 100 mL of water were added into a reaction bottle. Next, after the collagen was dissolved into the water, HCl aqueous solution (6N) was added into the reaction bottle, obtaining a solution with a pH lower than 3. Next, 0.5 g of collagen (strip-shaped fiber with a fiber length about 15 mm) was added into the reaction bottle. The weight ratio of the collagen and hydrophilic biodegradable polymer was 4:1. Next, after the collagen was dissolved into the water, the solution was injected into a two-dimensional mold and dried at room temperature. Next, the result was disposed in a chamber under the saturated vapor pressure of formaldehyde for 1 hour to undergo a cross-linking reaction, obtaining a film.

### Example 6

First, 0.5 g of polyvinylpyrrolidone (PVP, with a molecular weight about 50,000-60,000), and 100 mL of water were added into a reaction bottle. Next, after the collagen was dissolved into the water, HCl aqueous solution (6N) was added into the reaction bottle, obtaining a solution with a pH lower than 3. Next, 0.5 g of collagen (strip-shaped fiber with a fiber length about 15 mm) was added into the reaction bottle. The weight ratio of the collagen and hydrophilic biodegradable polymer was 1:1. Next, after the collagen was dissolved into the water, the solution was injected into a two-dimensional mold and dried at room temperature. Next, the result was disposed in a chamber and irradiated by an ultraviolet light with a wavelength of 254 nm and an intensity of 3 mW/cm² for 1 hour to undergo a cross-linking reaction, obtaining a film (VI).

### Example 7

First, 0.5 g of polyvinylpyrrolidone (PVP, with a molecular weight about 50,000-60,000), and 100 mL of water were added into a reaction bottle. Next, after the collagen was dissolved into the water, HCl aqueous solution (6N) was added into the reaction bottle, obtaining a solution with a pH lower than 3. Next, 0.5 g of collagen (flocculent fiber with a maximum length lower than 1.5 mm) was added into the reaction bottle. The weight ratio of the collagen and hydrophilic biodegradable polymer was 1:1. Next, after the collagen was dissolved into the water, the solution was injected into a two-dimensional mold and dried at room temperature. Next, the result was disposed in a chamber under the saturated vapor pressure of formaldehyde for 1 hour to undergo a cross-linking reaction, obtaining a film (VII).

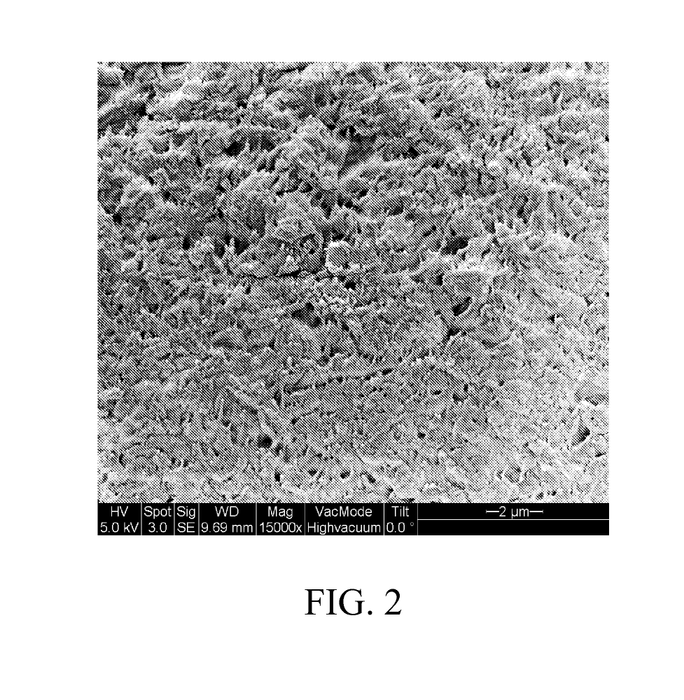

The film (VII) was observed by a scanning electron microscope, and the result is shown in FIG. 2. As shown in FIG. 2, the film (VII) does not have a multi-layered structure since the collagen has flocculent fibers with a maximum length lower than 1.5 mm.

### Example 8

First, 0.5 g of polyvinylpyrrolidone (PVP, with a molecular weight about 50,000-60,000), and 100 mL of water were added into a reaction bottle. Next, after the collagen was dissolved into the water, HCl aqueous solution (6N) was added into the reaction bottle, obtaining a solution with a pH lower than 3. Next, 0.5 g of collagen (gel-type) was added into the reaction bottle. The weight raio of the collagen and hydrophilic biodegradable polymer was 1:1. Next, after the collagen was dissolved into the water, the solution was injected into a two-dimensional mold and dried at room temperature. Next, the result was disposed in a chamber under the saturated vapor pressure of formaldehyde for 1 hour to undergo a cross-linking reaction, obtaining a film (VIII).

### Example 9

First, 0.5 g of polyvinyl alcohol (PVA, with a molecular weight about 30,000-50,000), and 100 mL of water were added into a reaction bottle. Next, after the collagen was dissolved into the water, HCl aqueous solution (6N) was added into the reaction bottle, obtaining a solution with a pH lower than 3. Next, 0.5 g of collagen (flocculent fiber with a maximum length lower than 1.5 mm) was added into the reaction bottle. The weight ratio of the collagen and hydrophilic biodegradable polymer was 1:1. Next, after the collagen was dissolved into the water, the solution was injected into a two-dimensional mold and dried at room temperature. Next, the result was disposed in a chamber under the saturated vapor pressure of formaldehyde for 1 hour to undergo a cross-linking reaction, obtaining a film (IX).

### Example 10

First, 0.5 g of polyethylene glycol/polyethylene oxide (PEG/PEO, with a molecular weight about 30,000-70,000), and 100 mL of water were added into a reaction bottle. Next, after the collagen was dissolved into the water, HCl aqueous solution (6N) was added into the reaction bottle, obtaining a solution with a pH lower than 3. Next, 0.5 g of collagen (flocculent fiber with a maximum length lower than 1.5 mm) was added into the reaction bottle. The weight ratio of the collagen and hydrophilic biodegradable polymer was 1:1. Next, after the collagen was dissolved into the water, the solution was injected into a two-dimensional mold and dried at room temperature. Next, the result was disposed in a chamber under the saturated vapor pressure of formaldehyde for 1 hour to undergo a cross-linking reaction, obtaining a film (X).

### Measurement of the Film

### Example 11

The light transmittance, swelling ratio, and suture pull-out strength of the films (I)-(X) of Examples 1-10 were measured, and the results are shown in Table 1.

The light transmittance of the film was determined by measuring the light absorption coefficient in the wavelength range of 350 nm to 700 nm of the film (having a saturated water content) via a spectrophotometer and measuring the light transmittance by means of the light absorption coefficient.

The swelling ratio of the film was measured by following steps. First, the weight of the dry film (W1) was measured. Next, the dry film was placed in water for 20 minutes, and then the weight of the swelling film (W2) was measured. Next, the swelling ratio was determined using the following equation:

$$swelling ratio = \frac{( W 2 - W 1 )}{W 1} .$$

In addition, the suture pull-out strength of the film was measured by the following steps. First, the film was cut into a test piece with a size of 20 mm×50 mm. After the test piece was placed in water for 20 minutes, a suture was threaded through the test piece (with a thickness between 50 μm and 500 μm when swelling with water) in a location wherein the distance between the location and a boundary of the test piece was 10 mm. The suture was pulled at about 10 mm/min via a tensile tester, thereby measuring the stress.

TABLE 1

| | collagen | | | | | suture | |
|---|---|---|---|---|---|---|---|
| | fiber | | | thickness | | pull-out | light |
| | length | | | (μm) | swelling | strength | Transmittance |
| | (mm) | polymer | treatment | (wet film) | ratio | (MPa) | (%) |
| film (I) | ~15 | — | formaldehyde | 150.1 | 6.7 | 29.0 | >85% |
| | | | gas | | | | |
| film (II) | ~15 | — | ultraviolet | 124.3 | 1.4 | 28.7 | >85% |
| | | | light | | | | |
| film (III) | ~15 | PVA | formaldehyde | 245.6 | 2.5 | 19.2 | >90% |
| | | (30,000- | gas | | | | |
| | | 50,000) | | | | | |
| film (IV) | ~15 | PEG | formaldehyde | 206.9 | 2.2 | 13.1 | >90% |
| | | (30,000- | gas | | | | |
| | | 70,000) | | | | | |
| film (V) | ~15 | PVP | formaldehyde | 223.7 | 9.5 | 20.0 | >95% |
| | | (50,000- | gas | | | | |
| | | 60,000) | | | | | |
| film (VI) | ~15 | PVP | ultraviolet | 185.1 | 2.1 | 8.8 | >85% |
| | | (50,000- | light | | | | |
| | | 60,000) | | | | | |
| film (VII) | flocculence/ | PVP | formaldehyde | 270 | 1.9 | 2.9 | >95% |
| | <1.5 | (50,000- | gas | | | | |
| | | 60,000) | | | | | |
| film (VIII) | gel-type | PVP | formaldehyde | — | — | — | opacity |
| | | (50,000- | gas | | | | |
| | | 60,000) | | | | | |
| film (IX) | flocculence/ | PVA | formaldehyde | — | — | — | <60% |
| | <1.5 | (30,000- | gas | | | | |
| | | 50,000) | | | | | |
| film (X) | flocculence/ | PEG | formaldehyde | — | — | — | <90% |
| | <1.5 | (30,000- | gas | | | | |
| | | 70,000) | | | | | |

As shown in Table 1, since the films (III)-(V) of Example 3-5 include the polymer (such as polyvinyl alcohol (PVA), polyethylene glycol/polyethylene oxide (PEG/PEO), or polyvinylpyrrolidone), the films (III)-(V) (swollen with water) have a greater light transmittance than 90%. In addition, due to the use of collagen with a fiber length that is longer than 15 mm, the film (III) (swollen with water) has a higher light transmittance than about 90%. In comparison, the films (IX) and (X) without the strip-shaped fiber have a light transmittance lower than 90%, even lower than 60%. The method for fabricating the film (V) has a cross-linking process different from that of the method for fabricating the film (VI). As shown in Table 1, the suture pull-out strength of the films (V) and (VI) of Examples 5 and 6 are both greater than 8 MPa. Moreover, since the collagen used in Example 5 has a longer fiber length than that used in Example 7, the film (V) has a multi-layered structure (as shown in FIG. 1) and has a suture pull-out strength of about 19.99 MPa. Due to the flocculent fiber, the film (VII) does not have a multi-layered structure (FIG. 2), and has a suture pull-out strength of about 2.9 MPa. Furthermore, due to the use of gel-type collagen rather than strip-shaped collagen fiber, the film (VIII) of Example 8 is opaque when swollen with water.

Accordingly, since the collagen used for preparing the composite material of the disclosure is strip-shaped fiber (having a fiber length between about 1.5 mm and 50 mm), the composite material of the disclosure can have a multi-layered structure. Therefore, the composite material exhibits a high swelling ratio, high suture pull-out strength, and high light transmittance. Furthermore, the composite material of the disclosure can be applied to wound dressing, ophthalmology, orthopedic implants, surgical use, drug delivery, or tissue engineering.

It will be apparent to those skilled in the art that various modifications and variations can be made to the disclosed methods and materials. It is intended that the specification and examples be considered as exemplary only, with a true scope of the disclosure being indicated by the following claims and their equivalents.

What is claimed is:

1. A composite material, which has a multi-layered structure constituted by a hydrophilic biodegradable polymer and a collagen, wherein the collagen is strip-shaped and has a fiber length between about 1.5 mm and 50 mm.

2. The composite material as claimed in claim 1, wherein there are at least ten stacked layers per 5 μm of thickness in the multi-layered structure.

3. The composite material as claimed in claim 2, wherein each stacked layer has a thickness between 0.1 to 1 μm.

4. The composite material as claimed in claim 1, wherein the hydrophilic biodegradable polymer comprises polyvinyl alcohol (PVA), polyethylene glycol/polyethylene oxide (PEG/PEO), polyvinylpyrrolidone (PVP), or a combination thereof.

5. The composite material as claimed in claim 1, wherein the hydrophilic biodegradable polymer has a molecular weight between 300 and 1,500,000.

6. The composite material as claimed in claim 1, wherein the weight ratio between the collagen and the hydrophilic biodegradable polymer is from 1:3 to 9:1.

7. The composite material as claimed in claim 1, wherein the composite material has a swelling ratio between 2 and 15.

8. The composite material as claimed in claim 1, wherein the composite material has a light transmittance larger than or equal to 90%.

9. The composite material as claimed in claim 1, wherein the composite material has a suture pull-out strength between 3 Mpa and 50 Mpa.

10. A composite material, comprising a product fabricated by the following steps:
a hydrophilic biodegradable polymer into a solvent, obtaining a first solution;
adjusting the pH value of the first solution to be lower than or equal to 5;
adding a collagen into the first solution, obtaining a second solution, wherein the collagen is strip-shaped and has a fiber length between 1.5 mm and 50 mm; and
subjecting the second solution to a drying process, obtaining a film.

11. The composite material as claimed in claim 10, wherein the drying process is a biaxial stretching process or solvent casting.

12. The composite material as claimed in claim 10, after the drying process, wherein the film is subject to a treatment, such that at least one of the hydrophilic biodegradable polymer and the collagen undergoes a cross-linking reaction.

13. The composite material as claimed in claim 12, wherein the treatment is performed in the presence of a cross-linking agent.

14. The composite material as claimed in claim 13, wherein the cross-linking agent comprises formaldehyde, glutaraldehyde, glyoxal, malondialdehyde, succinyl dialdehyde, phthalaldehyde, dialdehyde starch, polyacrolein, polymethacrolein, or a combination thereof.

15. The composite material as claimed in claim 12, wherein the treatment is performed by irradiating the film with a radiation.

16. The composite material as claimed in claim 15, wherein the radiation comprises ultraviolet light, or a Gamma ray.

17. The composite material as claimed in claim 10, wherein the hydrophilic biodegradable polymer comprises polyvinyl alcohol, polyethylene glycol/polyethylene oxide, polyvinylpyrrolidone, or a combination thereof.

18. The composite material as claimed in claim 10, wherein the hydrophilic biodegradable polymer has a molecular weight between 300 and 1,500,000.

19. The composite material as claimed in claim 10, wherein the weight ratio between the collagen and the hydrophilic biodegradable polymer is from 1:3 to 9:1.

20. The composite material as claimed in claim 10, wherein the composite material has a swelling ratio between 1 and 15.

21. The composite material as claimed in claim 10, wherein the composite material has a light transmittance larger than or equal to 90%.

22. The composite material as claimed in claim 10, wherein the composite material has a suture pull-out strength between 3 Mpa and 50 Mpa.
